(19) United States
(12) Patent Application Publication
(10) Pub. No.: US 2014/0221964 A1
(43) Pub. Date: Aug. 7, 2014

(54) SYSTEMS AND METHODS FOR LOCAL DRUG DELIVERY TO KIDNEYS

(71) Applicants: Yong-Fu Xiao, Mounds View, MN (US); Lepeng Zeng, Maple Grove, MN (US)

(72) Inventors: Yong-Fu Xiao, Mounds View, MN (US); Lepeng Zeng, Maple Grove, MN (US)

(21) Appl. No.: 14/008,489

(22) PCT Filed: Mar. 27, 2012
(86) PCT No.: PCT/US2012/030748

§ 371 Date: Apr. 1, 2014

Related U.S. Application Data
(60) Provisional application No. 61/468,057, filed on Mar. 27, 2011.
(60) Provisional application No. 61/468,059, filed on Mar. 27, 2011.

(51) Int. Cl. A61M 5/142; A61M 5/172

(52) U.S. Cl. CPC A61M 5/14276; A61M 5/1723
USPC 604/504; 604/66

(57) ABSTRACT

Drug-delivery systems for local drug delivery to kidneys and associated systems and methods are disclosed herein. One aspect of the present technology is directed to drug-delivery systems that include a physiological sensor, an implantable medical device, and a control module configured to communicate with the physiological sensor and to control delivery of a drug in response to a physiological parameter measured by the physiological sensor. The implantable medical device can be configured to be surgically implanted in a patient with a delivery opening at or near a renal capsule of a kidney of the patient. Suitable drugs for local delivery to a kidney can include diuretics, aldosterone antagonists, vasodilators, renin inhibitors, and combinations thereof. In some embodiments, local drug delivery to a kidney can be used to treat hypertension, heart failure, or another condition associated with renal activity.

### CROSS-REFERENCE TO RELATED APPLICATIONS

This application claims the benefit of U.S. Provisional Application No. 61/468,057, filed Mar. 27, 2011, and U.S. Provisional Application No. 61/468,059, filed Mar. 27, 2011, which are incorporated herein by reference in their entireties. Further, components and features of embodiments disclosed in the applications incorporated herein by reference may be combined with various components and features disclosed and claimed in the present application.

### TECHNICAL FIELD

The present technology relates generally to local drug delivery to kidneys. In particular, several embodiments are directed to local drug delivery to kidneys via implantable devices.

### BACKGROUND

Renal activity can affect a wide variety of organ-specific and systemic conditions. For example, renal activity can directly or indirectly affect conditions that are primarily renal (e.g., kidney stones) as well as conditions that are primarily non-renal (e.g., heart failure) or systemic (e.g., hypertension). In some cases, renal activity can affect primarily non-renal or systemic conditions via the renin-angiotensin-aldosterone system (RAAS). For example, as part of the RAAS, renal arterial constriction and corresponding renal hypoperfusion can cause excessive renal secretion of renin, which signals the body to retain sodium and water. Furthermore, renal sympathetic hyperactivity or overactivity can contribute to systemic sympathetic hyperactivity or overactivity. Retention of sodium and water and systemic sympathetic hyperactivity or overactivity can be key features of hypertension, heart failure, and a variety of other conditions.

Many conventional pharmacologic treatments derive their therapeutic effect entirely or partially by targeting renal activity. Such treatments can be intended to reduce and/or counteract renal contributions to conditions. For example, beta blockers can be used to reduce renin release and diuretics can be used to counteract sodium and water retention. Conventional pharmacologic treatments targeting renal activity, however, can have significant limitations, including limited efficacy, compliance issues, side effects, and others. Accordingly, there is a need for alternative treatment strategies, including alternative treatment strategies for reducing and/or counteracting renal contributions to conditions. Considering the pervasiveness and severity of hypertension, heart failure, and other conditions affected by renal activity, such alternative treatment strategies have the potential to dramatically impact public health.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology.

FIGS. 1-5 are partially schematic, cross-sectional views illustrating local drug delivery to a kidney in accordance with embodiments of the present technology.

FIG. 6 is a partially schematic diagram illustrating a drug-delivery system implanted within the body of a patient in accordance with an embodiment of the present technology.

FIG. 7 is a block diagram illustrating a selected configuration of the drug-delivery system shown in FIG. 6.

FIGS. 8-14 are partially schematic diagrams illustrating catheters and associated structures configured in accordance with embodiments of the present technology.

FIG. 15 is a block diagram illustrating a feedback algorithm executable for monitoring blood pressure and delivering drugs to kidneys in accordance with an embodiment of the present technology.

### DETAILED DESCRIPTION

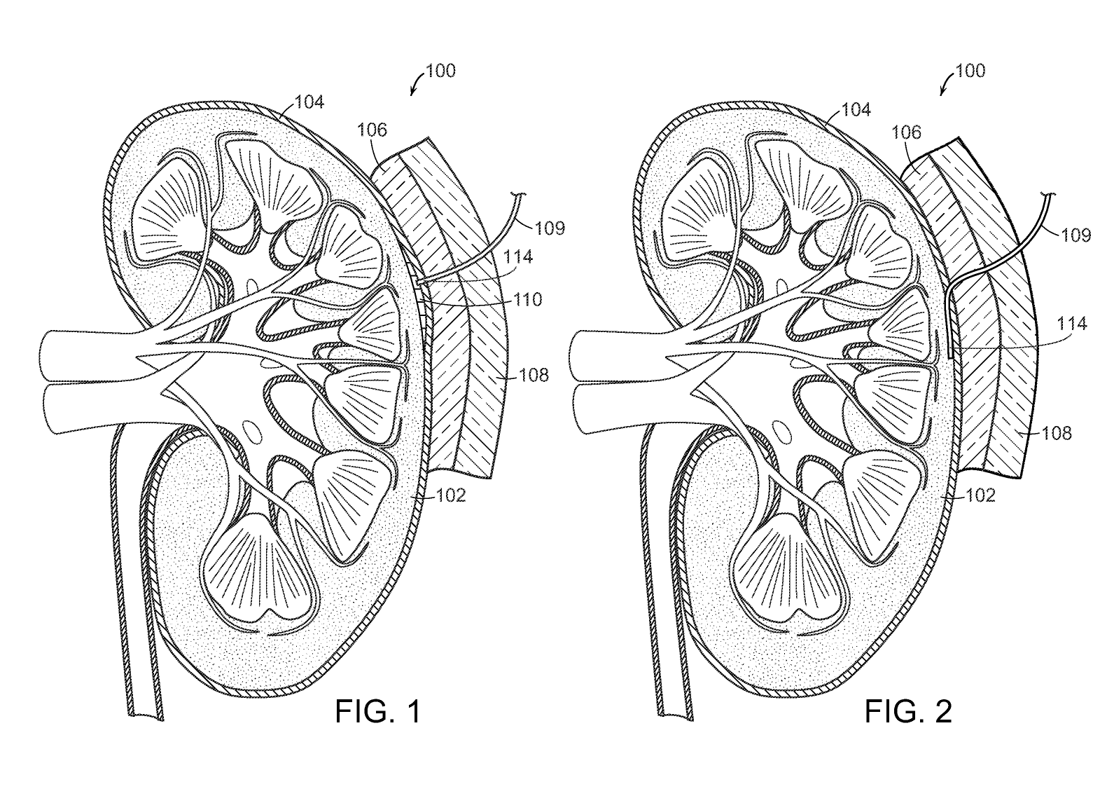

The present technology is generally directed to local drug delivery to kidneys and associated systems and methods. Specific details of several embodiments of the present technology are described herein with reference to FIGS. 1-15. Other embodiments of the present technology can have different configurations, components, or procedures than those described herein. For example, other embodiments can include additional elements and features beyond those described herein or be without several of the elements and features shown and described herein. For ease of reference, throughout the present disclosure identical reference numbers are used to identify similar or analogous components or features, but the use of the same reference number does not imply that the components or features should be construed to be identical. Indeed, in many examples described herein, the identically numbered parts are distinct in structure and/or function. Generally, unless the context indicates otherwise, the terms “distal” and “proximal” within the present disclosure reference a position relative to a reservoir or source (e.g., a drug reservoir). For example, “proximal” can refer to a position closer to a reservoir or source, and “distal” can refer to a position that is more distant from a reservoir or source. The headings provided herein are for convenience only.

### Selected Examples of Local Drug Delivery

Conventionally, drugs affecting renal activity are delivered systemically (e.g., ingested or administered intravascularly). While systemically delivered drugs may desirably affect renal activity to some degree, they typically have undesirable side effects (e.g., undesirable systemic effects and/or non-renal organ effects). Undesirable side effects can limit the usefulness of pharmacologic treatments. For example, with some drugs or dosages, the harm associated with undesirable side effects can outweigh the beneficial effect on renal activity, which can cause the drugs or dosages to be useless for most patients. In some embodiments of the present technology, drugs can be delivered locally to one or two kidneys of a patient. In contrast to conventional systemic drug delivery, local drug delivery to kidneys can reduce side effects by reducing (e.g., generally eliminating) systemic and/or non-renal exposure to the drugs. Furthermore, compared to systemically delivered drugs, locally delivered drugs can have the same or greater therapeutic effect in lower quantities and/or concentrations. Among other benefits, the disclosed techniques are expected to reduce drug cost and facilitate automatic drug delivery via implantable medical devices.

FIG. 1 is a partially schematic, cross-sectional view illustrating local drug delivery to a kidney 100 of a human patient in accordance with an embodiment of the present technology. As shown in FIG. 1, the kidney 100 can include a cortex 102 and a renal capsule 104 around the cortex 102. An adipose layer 106 and a renal fascia 108 can extend around the renal capsule 104. For clarity of illustration, only portions of the adipose layer 106 and the renal fascia 108 are shown. In some embodiments, a catheter 109 can be positioned to extend through the renal fascia 108, the adipose layer 106, and the renal capsule 104 to a potential space 110 of the kidney 100 between the cortex 102 and the renal capsule 104. The potential space 110 is shown with an exaggerated size in FIG. 1 for clarity of illustration. The catheter 109 can penetrate the renal capsule 104, for example, through an entry opening (e.g., a puncture, slit, or another suitable opening) in the renal capsule 104. The catheter 109 can include a distal portion 114 terminating at a delivery opening (not shown) within the potential space 110. In other embodiments, the catheter 109 can have other suitable configurations and/or positions relative to the renal capsule 104.

In FIG. 1, the cortex 102 and the renal capsule 104 are shown spaced apart around the potential space 110. In some cases, however, the potential space 110 can be within the cortex 102 and/or the renal capsule 104. Furthermore, the potential space 110 can be anatomically indistinguishable from the cortex 102 and/or the renal capsule 104. Delivering a drug into the potential space 110 can stretch the renal capsule 104, compress the cortex 102, and/or separate the cortex 102 from the renal capsule 104. Such structural effects can occur, for example, when the drug is delivered at a rate greater than a rate at which the drug can diffuse outward from a delivery location around the delivery opening. In other embodiments, delivering a drug into the potential space 110 can have little or no structural effect on the kidney 100. For example, the drug can be delivered at a rate equal to or less than a rate at which the drug can diffuse outward from the delivery location.

The potential space 110 can be relatively interconnected and permeable throughout the periphery of the kidney 100. Accordingly, in some embodiments, a drug delivered at one or more delivery locations within the potential space 110 can readily diffuse around and into the cortex 102 to therapeutically affect cells of the cortex 102. The potential space 110 can be entirely or mostly extravascular, which can reduce vascular transport of the drug away from the kidney 100. In some embodiments, local drug delivery can occur with little or no blood-to-catheter contact. Furthermore, the renal capsule 104, the adipose layer 106, or the renal fascia 108 can at least partially contain the drug from outward diffusion. For example, the renal capsule 104, the adipose layer 106, or the renal fascia 108 can be less permeable to diffusion of the drug than the cortex 102. This is not necessary for localization of drug delivery, however, because mere proximity of a delivery location to the cortex 102 can reduce systemic and/or non-renal exposure to the drug relative to systemic drug delivery. Accordingly, in some embodiments, the renal capsule 104, the adipose layer 106, and/or the renal fascia 108 can be equally permeable to diffusion of a drug as the cortex 102 or can be more permeable to diffusion of a drug than the cortex 102. The permeability of the cortex 102 relative to the renal capsule 104, the adipose layer 106, or the renal fascia 108 can depend on the properties (e.g., molecular weight and polarity) of the drug. For example, the adipose layer 106 can facilitate containment of relatively hydrophilic and/or polar drugs to a greater extent than relatively hydrophobic and/or non-polar drugs.

FIG. 2 is a partially schematic, cross-sectional view illustrating local drug delivery to the kidney 100 in accordance with another embodiment of the present technology. As shown in FIG. 2, the catheter 109 can extend along an inside wall of the renal capsule 104 through a potential space (not identified in FIG. 2) generally indistinguishable from a peripheral portion of the cortex 102. Increasing the length of the catheter 109 within the renal capsule 104 can increase a distance between the delivery opening and the entry opening in the renal capsule 104. This separation, alone or in combination with sealing the entry opening around the catheter 109 (e.g., using a balloon), can reduce outflow of a drug though the entry opening. Furthermore, in some embodiments, the orientation of the catheter 109 shown in FIG. 2 can cause the drug to be expelled from the delivery opening in a direction generally parallel to the inner wall of the renal capsule 104, which can enhance distribution of the drug around the cortex 102.

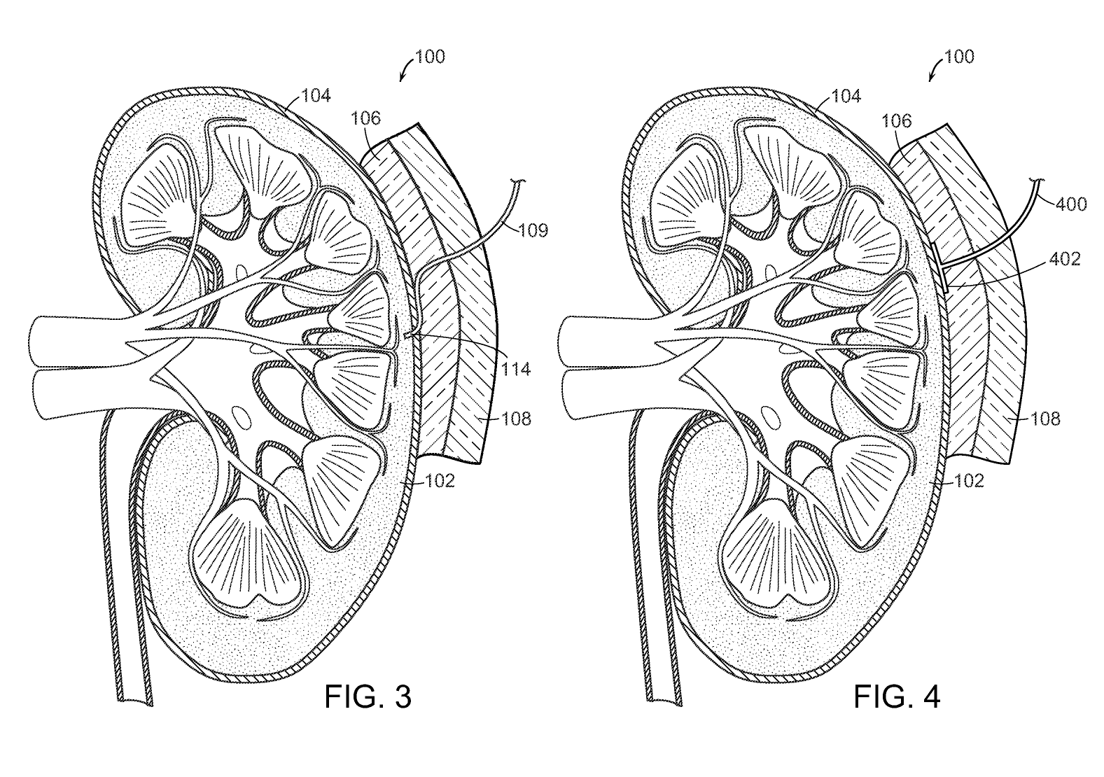

FIG. 3 is a partially schematic, cross-sectional view illustrating local drug delivery to the kidney 100 in accordance with another embodiment of the present technology. As shown in FIG. 3, the catheter 109 can extend along an outside wall of the renal capsule 104 before extending through the entry opening in the renal capsule 104 and into the potential space. In this configuration, the catheter 109 can be well positioned for attachment to the outside wall the renal capsule 104 (e.g., using stitches or another suitable surgical attachment mechanism). The renal capsule 104 typically is relatively tough and durable and can provide a secure anatomical anchor for the catheter 109. In other embodiments, the catheter 109 can be attached to the renal fascia 108, which is typically also relatively tough and durable. Furthermore, in some embodiments, the catheter 109 can be attached to the renal capsule 104 and the renal fascia 108 for even greater stability within the body. Such stability can be particularly useful when the catheter 109 is a portion of an implantable medical device intended to remain within the body for an extended period of time (e.g., years).

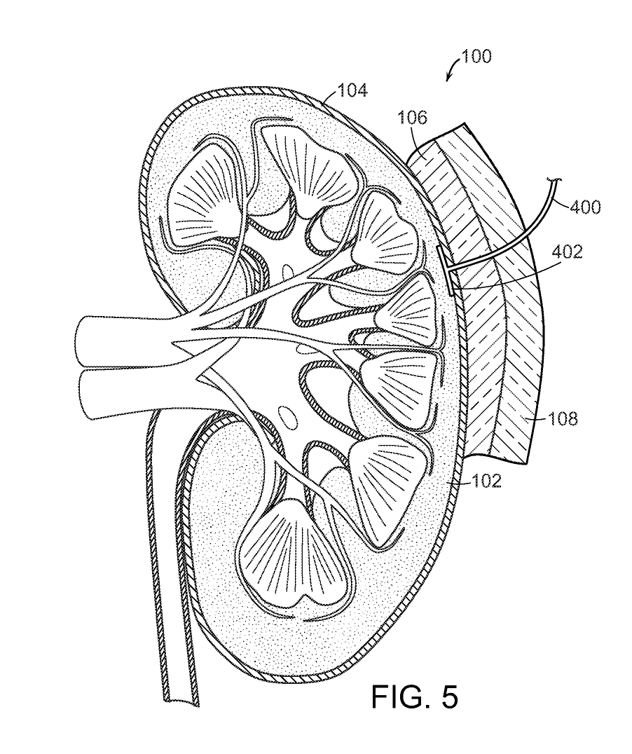

FIGS. 4 and 5 are partially schematic, cross-sectional views illustrating local drug delivery to the kidney 100 in accordance with additional embodiments of the present technology. As shown in FIGS. 4 and 5, a catheter 400 can extend through the renal fascia 108 and the adipose layer 106 and can include a diffusion patch 402. In some embodiments, the diffusion patch 402 is configured to preferentially deliver a drug through one major surface (e.g., a distal major surface) over another major surface. The diffusion patch 402 can have a variety of suitable positions within the renal anatomy. For example, as shown in FIG. 4, the diffusion patch 402 can be between the renal capsule 104 and the adipose layer 106 and can be configured to direct delivery of a drug through the renal capsule 104 toward the cortex 102. This positioning can be useful, for example, when a drug to be delivered readily diffuses through the renal capsule 104 and/or when surgically penetrating the renal capsule 104 is not desirable. As shown in FIG. 5, in other embodiments, the diffusion patch 402 can be between the renal capsule 104 and the cortex 102 and can be configured to direct diffusion of a drug directly into the cortex 102. This positioning can be useful, for example, when a drug to be delivered does not readily diffuse through the renal capsule 104 and/or when surgically penetrating the renal capsule 104 is acceptable.

The positions of the diffusion patch 402 shown in FIGS. 4 and 5 can have different advantages with respect to attaching the catheter 109 to the renal capsule 104. For example, in the arrangement shown in FIG. 4, the diffusion patch 402 can be relatively accessible for stitching to the outside wall of the renal capsule 104. In the arrangement shown in FIG. 5, a greater size of the diffusion patch 402 relative to the entry opening in the renal capsule 104 can entirely or partially secure the diffusion patch 402 within the renal capsule 104, and thereby anchor the catheter 109. In some embodiments, the diffusion patch 402 can be flexible and configured to move between collapsed and expanded states. For example, the diffusion patch 402 can be configured to be in a collapsed state when extended through the entry opening and to expand (e.g., spring) into an expanded state once inside the renal capsule 104. In still other embodiments, the diffusion patch 402 may have a different configuration and/or a different arrangement relative to the renal capsule 104.

### Selected Examples of Drugs

Locally delivered drugs in accordance with embodiments of the present technology can include any suitable pharmacological or therapeutic agent alone or in combination with one or more other such agents, one or more carrier materials (e.g., solutes or dispersion media), and/or one or more other suitable materials. The drugs can be liquids, solids, solutions, colloids, or have other suitable forms. In some embodiments, the drugs can he selected to affect renal activity (e.g., via the RAAS) when delivered locally to renal tissue. For example, the drugs can include RAAS-suppressing drugs therapeutically effective for treating hypertension, heart failure, or another condition associated with renal activity. Treatable conditions can also include renal conditions (e.g., kidney stones, kidney infection, and kidney cancer, among others).

Examples of suitable functional classes of drugs include diuretics, aldosterone II receptor antagonists, vasodilators, calcium-channel blockers, renin inhibitors, nerve inhibitors, local anesthetics, angiotensin II receptor blockers, ACE inhibitors, anti-inflammatories, antibiotics, endothelin-receptor antagonists, and alpha-2 receptor agonists, among others. Examples of suitable drugs and drug types include bumetanide, furosemide, natriuretic peptides (e.g., atrial natriuretic peptides, brain natriuretic peptides, and C-type natriuretic peptides), spironolactone, eplerenone, isosorbide, isosorbide dinitrate, isosorbide-5-mononitrate, apresoline, aliskiren (e.g., TEKTURNA aliskiren), chlorothiazide (e.g., DIURIL chlorothiazide), indapamide, lidocaine, procaine, hypertonic solutions (e.g., high-concentration NaCl), amlodipine (e.g., NORVASC amlodipine), losartan (e.g., HYZAAR losartan potassium and hydrochlorothiazide), bosentan, clonidine (e.g., CATAPRES clonidine), enalapril, lisinopril, captopril, carvedilol, metoprolol, bisoprolol, nitric oxide (NO), compounds that are capable of generating NO in situ (e.g., glyceryl trinitrate, isoamyl nitrite, sodium nitroprusside, molsidomine, S-nitrosoglutathione, and other suitable NO-donor compounds), antibodies, peptides, siRNAs, and polynucleotides that encode polypeptides that affect renal activity, among others.

### Selected Examples of Drug-Delivery Systems

Drug-delivery systems in accordance with embodiments of the present technology can be configured for local drug delivery to one or two kidneys of a patient as described above or in another suitable manner. In some embodiments, the systems can be configured to deliver drugs as needed in response to a condition (e.g., hypertension, heart failure, or another condition affected by renal activity). For example, drug delivery can occur in real time, near real time, or after a suitable delay in response to a metric corresponding to the condition. Moreover, the systems can be configured to deliver drugs in a manner that can limit systemic residence of the drugs and thereby limit systemic side effects associated with the drugs.

In some embodiments, a drug-delivery system can include an implantable drug reservoir, a pump (e.g., an osmotic pump), and at least one catheter configured to locally deliver a drug proximate (e.g., into) the renal capsule. The system can he used to suppress the RAAS to treat hypertension, heart failure, or another condition affected by renal activity. The catheter connecting to the reservoir can be placed into the renal capsule of one or two kidneys of a patient and the pump can be programmed to adjust the rate of drug infusion. The system can further provide closed-loop feedback control via a sensor (e.g., a blood-pressure sensor), which can be implanted or not implanted. Examples of suitable implantable systems include the ISOMED and SYNCHROMED II implantable devices commercially available from Medtronic, Inc. (Minneapolis, Minn.), among others. Further examples are shown in U.S. Pat. No. 4,692,147, which is incorporated herein by reference in its entirety.

Drug-delivery systems configured in accordance with embodiments of the present technology can include a reservoir configured to store a drug, a pump configured to infuse the drug according to a desired infusion mode and/or rate, and a catheter configured to route the drug from the pump to a desired anatomical site. Some embodiments can include multiple reservoirs configured to store the same or different drugs at the same or different concentrations. Furthermore, the systems can include a plurality of catheters, individually configured for delivery of drugs from different reservoirs and/or for delivery of drugs to different locations (e.g., to different locations within the renal capsule of a single kidney or to different kidneys). In some embodiments, the system or a portion thereof can be implanted in anatomical proximity to a kidney. The system or a portion thereof, however, also can be implanted at a distance from the kidney. The catheter can be of a length sufficient to traverse the distance from the anatomical location of the reservoir, once implanted, to the kidney and to provide a guided pathway for a drug from the reservoir to the kidney. In some embodiments, the catheter can be flexible to permit individualized routing through the anatomy of the patient.

The system can be configured to permit long-term (e.g., lifetime) treatment for hypertension, heart failure, or another condition affected by renal activity. For example, the system can be configured to deliver drugs at a desired rate over long durations without intervention, and to make drug therapy much easier and more accurate relative to other treatments. The system or a portion thereof can be implanted subcutaneously (e.g., in the chest, abdominal cavity, or another suitable anatomical location). In some embodiments, the system can include a self-sealing, needle-penetrable septum configured to be implanted subcutaneously (e.g., directly beneath the skin). The septum can provide a fluid passageway configured to permit the reservoir to be refilled periodically via a transcutaneous injection. Accordingly, the reservoir can be filled or refilled without requiring surgical removal from the patient's body and without requiring any other significant surgical procedure.

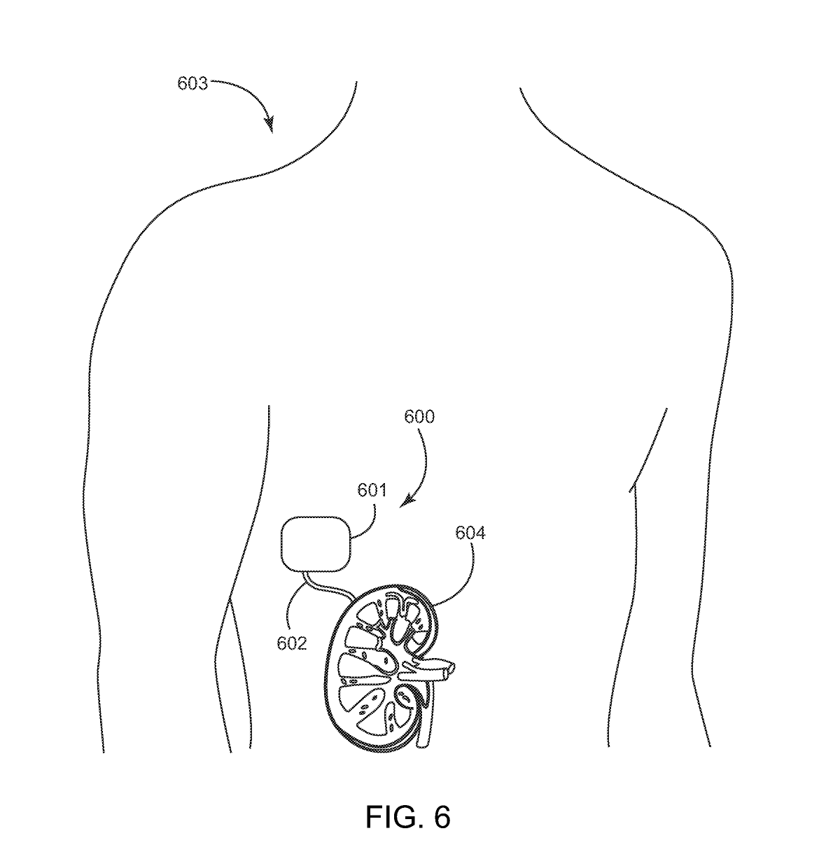

The reservoir can include a discharge outlet through which the drug can be directed during delivery. The outlet can be connected to a catheter (e.g., a flexible medical tubing) leading to the targeted delivery site. The system can further include a power source, a pump, and associated electronics configured to control delivery of the drug to the patient in response to a physiological measurement. Referring to FIG. 6, for example, a drug-delivery system 600 configured in accordance with an embodiment of the technology can include a reservoir 601 and a catheter 602. The system 600 can be surgically implanted subcutaneously in the pectoral or abdominal region of the body of a patient 603 with the catheter 602 extending between the reservoir 601 and a renal capsule 604. The system 600 can include any suitable mechanism capable of delivering one or more drugs to the renal capsule 604. For example, the system 600 can include a pumping mechanism (not shown) and a power supply (not shown) configured to operate the system 600 such that the drug is delivered to the renal capsule 604 at a predetermined infusion rate. It should be understood that some pumps used in connection with the system 600, however, may not require a separate power supply.

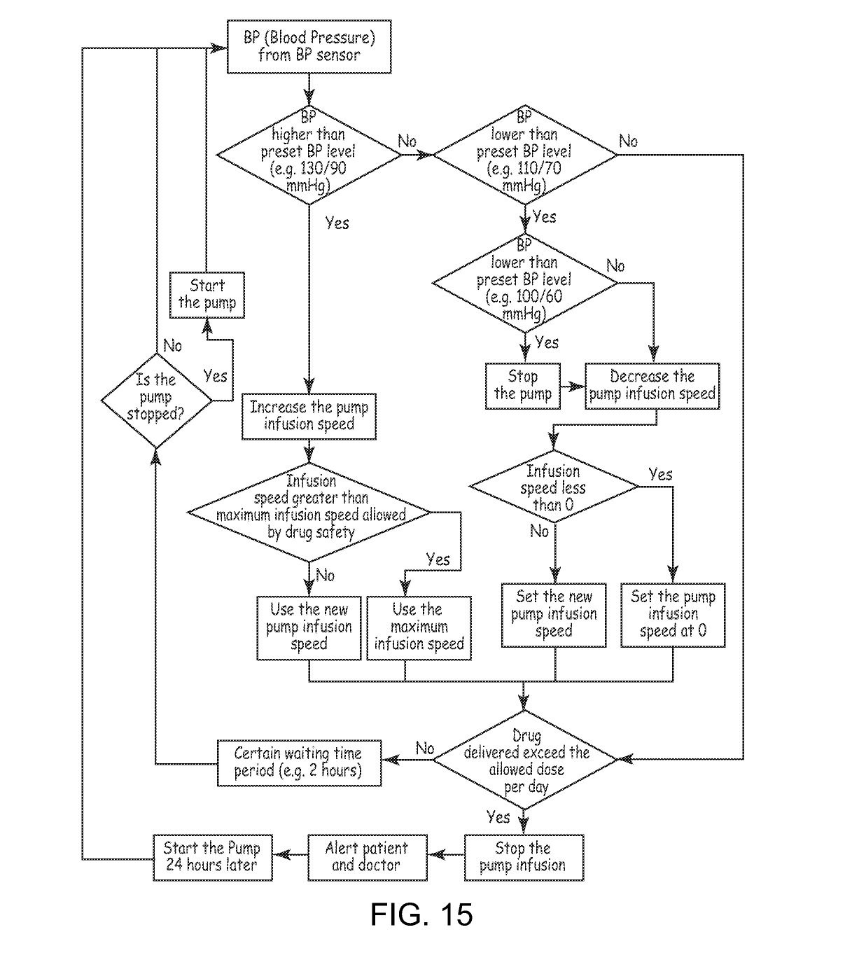

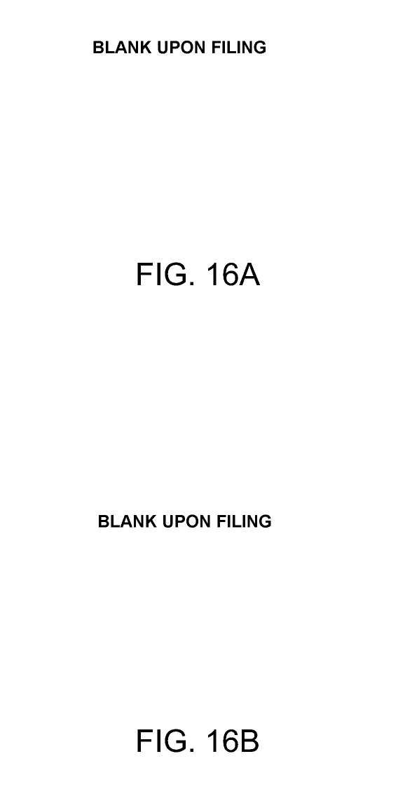

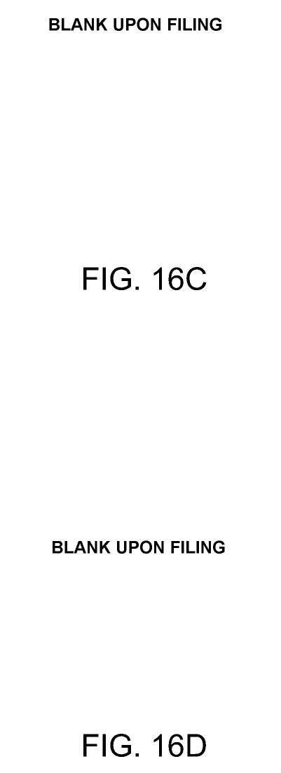

While the system 600 is shown in FIG. 6 as implantable, it should be understood that the system 600 can be either implanted or extracorporeal. As used herein, the term “implantable” means that a system, apparatus, or device is adapted for implantation in the body of patient where it is primarily or entirely subcutaneous. An extracorporeal system 600 may be appropriate in instances where, for example, short-term therapy is indicated and, therefore, implanting the system 600 may not be required. Furthermore, while FIG. 6 shows the system 600 delivering one or more drugs to the renal capsule 604, the one or more drugs can be directly delivered to one or more renal arteries, to other kidney tissue (e.g. the cortex), to the fat capsule around the kidney, and/or to other suitable portions of the renal anatomy. The system 600 can be configured to deliver one or more drugs via an automated control algorithm (e.g., the algorithm described below with reference to FIG. 15 or another suitable algorithm) and/or under the control of a clinician.

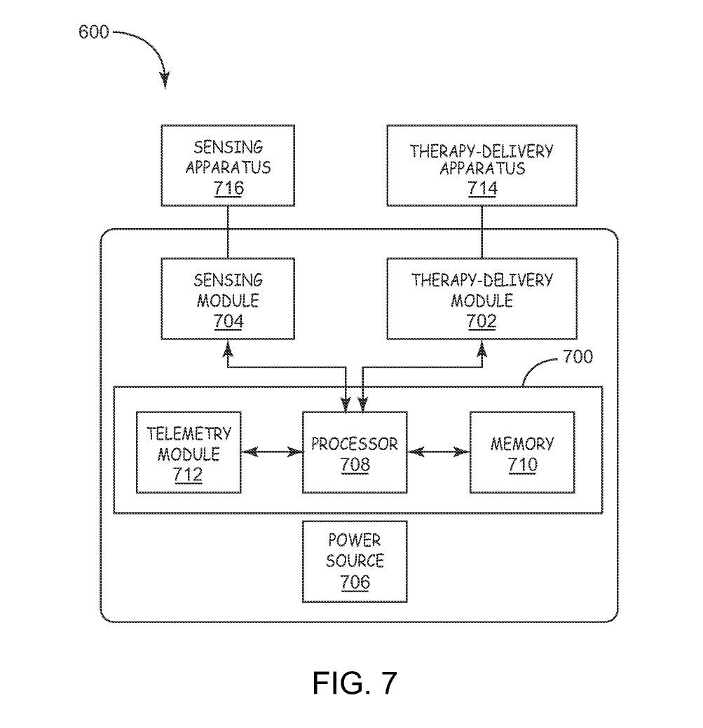

FIG. 7 is a block diagram illustrating a selected configuration of the system 600. As shown in FIG. 7, the system 600 can include a control module 700, a therapy-delivery module 702 (e.g., a pump and the reservoir 601), a sensing module 704, and a power source 706. The control module 700 can include a processor 708, memory 710, and a telemetry module 712. The memory 710 can include computer-readable instructions that, when executed (e.g., by the processor 708) cause the system 600 or one or more of its components (e.g., the control module 700) to perform various functions attributed to the system 600 or the component(s) as described herein. For example, the computer-readable instructions can cause the processor 708 to execute the algorithm described below with reference to FIG. 15 or another suitable algorithm. The memory 710 can include any volatile, non-volatile, magnetic, optical, or electrical media, such as random access memory (RAM), read-only memory (ROM), non-volatile RAM (NVRAM), electrically-erasable programmable ROM (EEPROM), flash memory, or any other suitable digital media.

The processor 708 of the control module 700 can include any one or more of a microprocessor, a controller, a digital signal processor (DSP), an application-specific integrated circuit (ASIC), a field-programmable gate array (FPGA), or equivalent discrete or integrated logic circuitry. In some embodiments, the processor 708 can include multiple components, such as any combination of one or more microprocessors, one or more controllers, one or more DSPs, one or more ASICs, or one or more FPGAs, as well as other discrete or integrated logic circuitry. The functions attributed to the processor 708 herein can be embodied as software, firmware, hardware, or any combination thereof. The processor 708 or any other portion of the control module 700 can employ digital signal analysis techniques to characterize the digitized signals stored in memory 710 (e.g., to recognize and classify the patient's blood pressure) employing any of the numerous signal processing methodologies known in the art.

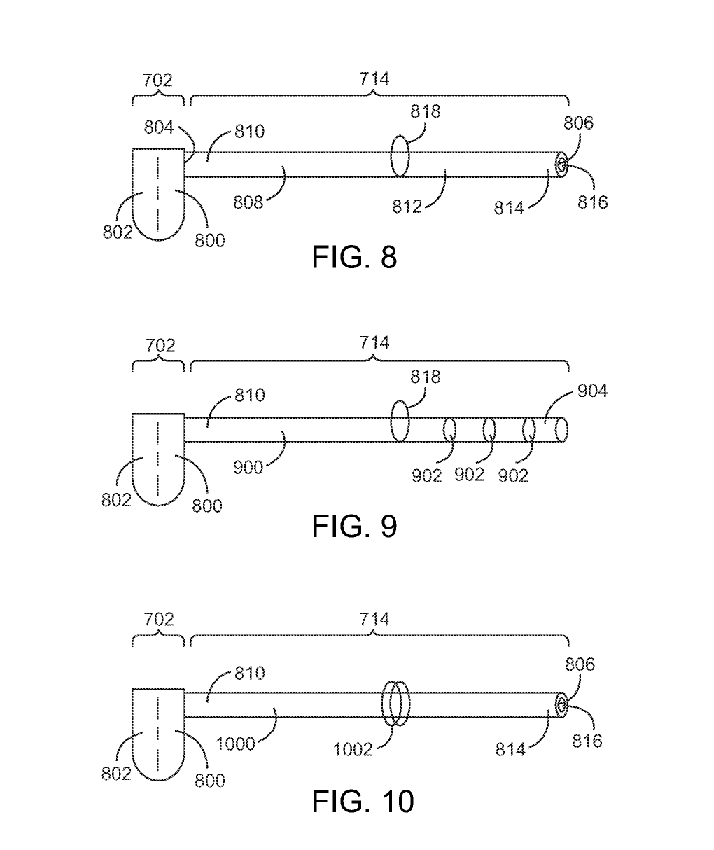

The control module 700 can be coupled to and control the therapy-delivery module 702, which can be configured to deliver therapy (e.g., RAAS-suppressing therapy) to the renal capsule 604 (FIG. 6) according to one or more therapy programs that can be stored in the memory 710. Specifically, the processor 708 of the control module 700 can control the therapy-delivery module 702 to deliver one or more drugs to the renal capsule 604 with the infusion rate, timing, duration, volume, and/or concentration specified by one or more therapy programs. The therapy-delivery module 702 can be coupled (e.g., electrically coupled) to a therapy-delivery apparatus 714 of the system 600 (FIG. 6) such that the therapy-delivery module 702 can use the therapy-delivery apparatus 714 to deliver therapy to the patient 603 (FIG. 6). The therapy-delivery apparatus 714 can include, among other therapy-delivery devices, a catheter 602 (e.g., having one or more of the features shown in FIGS. 8-14). The therapy-delivery module 702 can be configured to generate and deliver drug therapy to the renal capsule 604. For example, the therapy-delivery module 702 can deliver one or more RAAS-suppressing drugs to the renal capsule 604 in response to changes in the patient's blood pressure detected by a sensing apparatus 716 of the system 600. In other embodiments, the sensing apparatus 716 can be configured to detect another suitable physiological parameter (e.g., heart rate).

The sensing apparatus 716 can be configured to monitor one or more physiological parameters continuously or periodically and to transmit data (e.g., signals) generating by the monitoring. The data can include, for example, single measurements, multiple measurements (e.g., at multiple times), averages, derivatives (e.g., rates of change), and/or other suitable representations of the one or more physiological parameters. The control module 700 can be configured to receive the data and to modify drug delivery based on the data. Furthermore, in some embodiments, the sensing apparatus 716, the control module 700, or another component of the system 600 can be configured to transmit the data via telemetry for remote monitoring of patient status.

The control module 700 can be coupled to and control the sensing module 704 to receive one or more signals from the sensing apparatus 716. The sensing module 704 can be coupled (e.g., electrically coupled) to the sensing apparatus 716 (e.g., to monitor signals from the sensing apparatus 716). The sensing apparatus 716 can include one or more implantable sensors and/or extracorporeal sensors. An implantable sensor can be placed in one or more locations of the body. For example, an implantable sensor can be placed in the pulmonary artery, the leg, or the arm. Additionally, an implantable sensor can be used to signal impedance which can be used as an index for heart contractility function. Examples of suitable implantable sensors include the OPTIVOL system commercially available from Medtronic, Inc. (Minneapolis, Minn.) and the ENDOSURE wireless pressure-measurement system commercially available from CardioMEMS, Inc. (Atlanta, Ga.), among others. Some implantable sensors can have a diameter of about 1 mm and can be placed, for example, directly into the femoral artery. Such a sensor can measure a patient's blood pressure rapidly (e.g., about 30 times per second). The sensor can be connected via a flexible micro-cable to a transponder unit, which can be likewise implanted in the patient 603. The sensor can be configured to digitize and encode data coming from a micro-sensor and transmit the data to the sensing module 704.

Examples of suitable blood-pressure sensors include sensors that measure systolic and/or diastolic blood pressure using the oscillometric technique. Such sensors can include, for example, adjustable cuffs, pump bulbs, and/or pressure transducers. In some embodiments, an extracorporeal blood-pressure sensor can include a cuff-less, wearable blood-pressure sensor. Such a sensor can be configured to provide continuous, 24-hour monitoring using pulse-wave velocity, which can allow blood pressure to be calculated by measuring the pulse at two points along an artery. In some embodiments, such a sensor can detect differences in blood pressure along two points in the hand and correct for variation stemming from the position of the hand relative to the heart. Regardless of the particular extracorporeal blood-pressure sensor used, data can be transmitted to the sensing module 704 via telemetry.

The telemetry module 712 can be operatively connected to the processor 708 to provide for communication between one or more of the components of the system 600 and, for example, an external user, an external programming device, etc. Telemetry-control devices, systems, and methods that can be used in connection with the methods and systems described herein are known to those of skill in the art. Examples of such telemetry devices include those described, for example, in U.S. Pat. No. 5,558,640 (Pfeiler et al.), U.S. Pat. No. 5,820,589 (Torgerson et al.), and U.S. Pat. No. 5,999,857 (Weijand et al.), which are all incorporated herein by reference in their entireties. Although the telemetry module 712 is shown connected to the processor 708 in FIG. 7, it will be understood that the telemetry module 712 can alternatively be connected directly to one or more components of the therapy-delivery module 702 (e.g., a pump). The telemetry module 712 can provide for one-way or two-way communication.

The therapy-delivery module 702 can include a plurality of reservoirs and/or pumps, as shown in FIG. 8-14. In embodiments that include multiple reservoirs, each reservoir can independently contain a different drug and the different drugs can be administered independently of one another. Similarly, in embodiments that include multiple pumps, each pump can be controlled independently of any other pump. One example of such an arrangement of components of a therapy-delivery module 702 and a therapy-delivery apparatus 714 of the system 600 is shown in FIG. 8. The therapy-delivery module 702 can include a reservoir 800 and a pump 802. The reservoir 800 can include an outlet 804 that provides fluid communication between the contents of the reservoir 800 and a lumen 806 of a catheter 808 of the therapy-delivery apparatus 714. The relative diameter of the catheter 808 is exaggerated for ease of illustration of the structure thereof and the full length of the catheter 808 is not shown for simplicity of illustration. A proximal end portion 810 of the catheter 808 can be coupled to the reservoir 800 at the outlet 804. The pump 802 can be operatively connected to the reservoir 800 so that the pump 802 can control dispensing of a drug from the reservoir 800 to the renal capsule 604 via the catheter 808.

The catheter 808 can include an elongated tubular portion 812 that extends from the proximal end portion 810 to a distal end portion 814 of the catheter 808. The lumen 806 can terminate at an opening 816 at the distal end portion 814. A drug can be delivered through the catheter 808 via the lumen 806 and exit the catheter 808 through the opening 816. The body of the catheter 808 can be constructed of any suitable structure or material (e.g., an elastomeric tube). Suitable materials include, but are not limited to, silicone rubber (e.g., polydimethyl siloxane) and polyurethane, both of which can provide good mechanical properties and are very flexible. Suitable materials for the catheter 808 can also be chemically inert so that the catheter 808 generally does not interact with a drug, body tissue, or body fluids over an extended time period. The diameter of the lumen 806 can be large enough to accommodate expected infusion rates with acceptable flow resistance. The wall of the catheter 808 can be thick enough to withstand normal handling during the implantation procedure and forces from body tissues during normal motion. As an example, in one particular embodiment the catheter 808 can have an outside diameter of about 1.25 mm and an inside diameter of about 0.5 mm, with a wall thickness of about 0.375 mm. In some embodiments, the catheter 808 can be about 50 cm long or another length selected to reach from the reservoir 800 to a patient's kidney. In other embodiments, the inside and outside diameters and the length of the catheter 808 can vary to meet the needs of implantation and/or drug infusion.

The catheter 808 can include a site for anchoring the catheter 808 so that the opening 816 can deliver a drug to the renal capsule. Generally, the site of attachment can be located proximal to the opening 816. In use, the opening 816 can be implanted into the space between the renal capsule and the renal cortex and anchored in place so that the drug exiting the opening 816 can generally be contained by the renal capsule. In the embodiment shown in FIG. 8, the catheter 808 can be anchored (e.g., to the renal capsule) using one or more sutures 818. As shown in FIG. 8, the opening 816 can be at the distal end portion 814 of the catheter 808. As a result, drugs delivered to the renal capsule through catheter 808 can exit through the opening 816 proximate the distal end portion 814. Many alternatives may be provided for the structure through which the drug can pass out of the catheter 808. Some alternatives are illustrated in FIGS. 9-14. In addition, FIGS. 9-14 illustrate various alternatives for anchoring the catheter 808. The device 600 can include combinations of embodiments where those alternatives are not incompatible with one another.

FIG. 9, for example, illustrates a section of an alternative design in which a catheter 900 includes multiple openings 902 formed through a wall of the catheter 900. As a drug moves through the lumen of the catheter 900, it can exit through one or more of the openings 902. In such an embodiment, it can be useful for a distal end portion 904 of the catheter 900 to be closed such that the drug can exit more readily through openings 902 in the catheter wall. However, in some embodiments, the distal end portion 904 can be open to permit the drug to exit through an opening (not shown) at the distal end portion 904 of the catheter 900. The size and spacing of the openings 902 can vary depending on a variety of factors (e.g., the viscosity of the drug to be delivered, the desired delivery rate, etc.). The site of attachment can be located proximal to the most proximal of the openings 902. Furthermore, in some embodiments, one or more of the openings 902 can be individually connected to separate supply lumens. In this way, drug delivery through the openings 902 can be selectively controlled. This feature can be useful, for example, to facilitate selective delivery of drugs to different locations within the renal anatomy and/or selective delivery of different drugs (e.g., different drug types and/or concentrations).

The axial length (e.g., as measured along an axis extending from the proximal to the distal end of the catheter 900) of the portion of the catheter 900 that includes the openings 902 can be selected based on a variety of factors. The length of the portion of the catheter 900 over which the openings 902 are dispersed can, in some embodiments, have a limited axial length (e.g., about 320 mm or less, about 160 mm or less, or about 120 mm or less). At relatively short lengths, it can be useful for the portion of the catheter 900 over which the openings 902 are dispersed to have an axial length of about 20 mm or more (e.g., about 40 mm or more). In embodiments that include a plurality of openings (e.g., those illustrated in FIGS. 9 and 11-14) each opening can have a similar diameter or an independently different diameter compared to the diameter of any other opening. For example, in some embodiments each opening can have a diameter of at least about 0.1 mm and no more than about 5 mm. Also, the pattern of the openings can be symmetrical along a given length (e.g., of a catheter or a catheter extension) or within a given area (e.g., of a patch or a mesh sheet). Alternatively, the pattern of openings can be asymmetrical so that, for example, openings and drug delivery can be concentrated in one or more desired locations. In still other embodiments, the catheter 900 may have different lengths and/or the openings may have different configurations.

FIG. 10 illustrates another embodiment in which a catheter 1000 can be anchored in place using a balloon 1002 located proximal to the opening 816. The balloon 1002 in an uninflated state can be situated deep in the renal capsule (e.g., between the renal capsule and the renal cortex) and then inflated, thereby anchoring the implanted catheter 1000. In some embodiments, the balloon 1002 can be inflated and/or deflated via a separate lumen extending axially within the catheter 1000. For example, such a lumen can be configured for connection to an inflation/deflation device (e.g., a syringe) at an extracorporeal location. In other embodiments, the balloon 1002 can be inflated with a drug via the same lumen that delivers the drug to the opening 816.

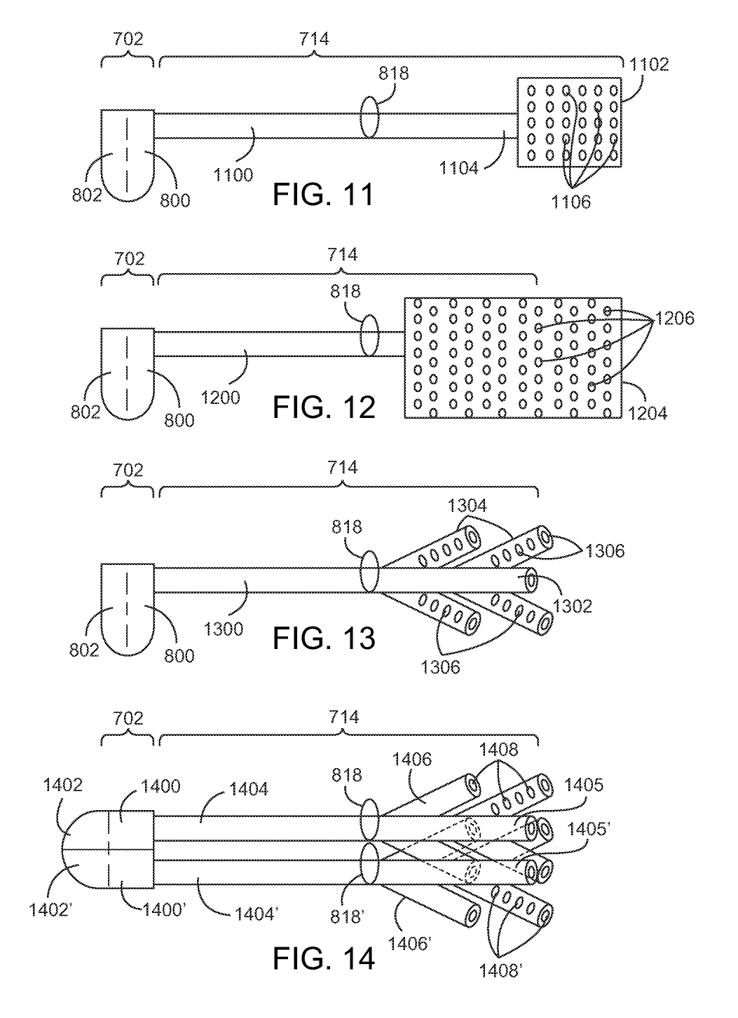

FIG. 11 illustrates another embodiment in which a catheter 1100 can include a drug-delivery patch 1102 appended to a distal end 1104 of the catheter 1100. The patch 1102 can include a plurality of openings 1106 through which one or more drugs can be dispensed. The patch 1102 can provide fluid communication between the lumen (not identified in FIG. 11) of the catheter 1100 at the distal end 1104 of the catheter 1100 and the openings 1106. In some embodiments, the patch 1102 can be implanted into the space between the renal capsule and the renal cortex and anchored in place using one or more sutures 818 (e.g., as described with reference to FIG. 8), a balloon (e.g., as described with reference to FIG. 10), or another suitable connector. The patch 1102 can provide additional surface area in which to provide the openings 1106 for infusion of the drug(s).

The dimensions of the patch 1102 can be generally sufficient to cover at least a portion of the surface area of the renal cortex. Thus, in some embodiments, the patch 1102 can be designed to cover, for example, at least 1%, at least 2%, at least 3%, at least 4%, at least 5%, at least 10%, at least 15%, at least 20%, at least 25%, at least 30%, at least 35%, at least 40%, at least 45%, at least 50%, at least 60%, at least 70%, at least 75%, at least 80%, at least 85%, at least 90%, at least 95%, at least 96%, at least 97%, at least 98%, at least 99%, or 100% of the renal cortex. In a particular embodiment, the patch 1102 can be designed so that it covers, for example, approximately 25% of the surface area of the renal cortex. The patch 1102 can, however, be designed to cover more or less of the renal cortex as may be appropriate for any contemplated therapy. Thus, the patch 1102 can have a surface area of at least 1 cm², for example, at least 2 cm², at least 3 cm², at least 4 cm², at least 5 cm², at least 10 cm², at least 15 cm², at least 20 cm², at least 25 cm², at least 30 cm², at least 40 cm², at least 50 cm², at least 60 cm², at least 70 cm², at least 80 cm², at least 90 cm², at least 100 cm², at least 110 cm², at least 120 cm², at least 130 cm², at least 140 cm², at least 150 cm², at least 160 cm², at least 170 cm², at least 180 cm², at least 190 cm², at least 200 cm², or another suitable size.

The patch 1102 can be constructed according to conventional methods for constructing drug-delivery patches. In some embodiments, for example, the patch 1102 can include two layers that form a reservoir therebetween, from which the drug(s) may be dispensed through the openings 1106. Such a reservoir can be in fluid communication with the reservoir 800 (e.g., through the lumen of the catheter 1100). In some embodiments, one layer of the patch 1102 can include a drug-permeable material that can be oriented in use to cover or otherwise face the renal cortex. In such embodiments, a drug can be dispensed through the drug-permeable material rather than, or in addition to, through openings 1106. In other embodiments, one or more layers of the patch 1102 can be constructed from drug-impermeable materials and, therefore, can limit diffusion, dispensing, or other delivery of the drug except, for example, through openings 1106 or through a drug-permeable layer.

FIG. 12 illustrates an alternative embodiment in which a catheter 1200 can include a mesh sheet 1204 appended to a distal end (not identified in FIG. 12) of the catheter 1200. The mesh sheet 1204 can include a plurality of openings 1206 through which one or more drugs can be dispensed. The mesh sheet 1204 can provide fluid communication between a lumen (not identified in FIG. 12) at the distal end of the catheter 1200 and the openings 1206. In some embodiments, the mesh sheet 1204 can be implanted into the space between the renal capsule and the renal cortex and anchored in place using one or more sutures 818 (e.g., as described with reference to FIG. 8), a balloon (e.g., as described with reference to FIG. 10), or another suitable connector. The mesh sheet 1204 can provide additional surface area in which to provide the openings 1206 for infusion of the drug(s).

The dimensions of the mesh sheet 1204 can be generally sufficient to cover at least a portion of the surface area of the renal cortex. Thus, in some embodiments, the mesh sheet 1204 can be designed to cover, for example, at least 1%, at least 2%, at least 3%, at least 4%, at least 5%, at least 10%, at least 15%, at least 20%, at least 25%, at least 30%, at least 35%, at least 40%, at least 45%, at least 50%, at least 60%, at least 70%, at least 75%, at least 80%, at least 85%, at least 90%, at least 95%, at least 96%, at least 97%, at least 98%, at least 99%, or 100% of the renal cortex. In a particular embodiment, the mesh sheet 1204 can be designed so that it covers, for example, approximately 25% of the surface area of the renal cortex. The mesh sheet 1204 can, however, be designed to cover more or less of the renal cortex as may be appropriate for any contemplated therapy. Thus, the mesh sheet 1204 can include a surface area of at least 1 cm², for example, at least 2 cm², at least 3 cm², at least 4 cm², at least 5 cm², at least 10 cm², at least 15 cm², at least 20 cm², at least 25 cm², at least 30 cm², at least 40 cm², at least 50 cm², at least 60 cm², at least 70 cm², at least 80 cm², at least 90 cm², at least 100 cm², at least 110 cm², at least 120 cm², at least 130 cm², at least 140 cm², at least 150 cm², at least 160 cm², at least 170 cm², at least 180 cm², at least 190 cm², at least 200 cm², or another suitable size. The mesh sheet 1204 can be constructed according to conventional methods for constructing mesh sheets. In some embodiments, the mesh sheet 1204 can be constructed of a biomaterial (e.g., collagen), a mixture of DL-lactic acid polymer and a copoly(L-lactic acid/delta-valerolactone) polymer, or any other material or combination of materials suitable for drug delivery.

FIG. 13 illustrates an embodiment in which a catheter 1300 can include a distal end portion 1302 having a plurality of extensions 1304. Each extension 1304 can include one or more openings 1306. The number of openings 1306 of each extension 1304 can vary independently of the number of openings 1306 of any other extension 1306. Alternative designs for increasing the number of openings are possible in addition to the patch, mesh sheet, and extensions described herein. An extension 1304 can be constructed of material that is the same as or similar to the material used to construct the catheter 1300. If appropriate, each extension 1304 can be constructed independently of the others and/or of a different material. As described above with reference to FIGS. 8-9, the distal end of the catheter 1300 and/or the distal ends of each extension 1304 can, independently of the others, be open (e.g., as shown in the distal end portion 814 of FIG. 13) or closed (e.g., as shown in the distal end portion 904 of FIG. 9).

The dimensions (e.g., total diameter, lumen diameter, and wall thickness) of the extensions 1304 can be similar to the dimensions of the catheter 1300. The axial length of each extension 1304 can vary according to the drug-delivery requirements of the intended therapy. Thus, the axial length of each extension 1304 can be as little as, for example, 1 mm or more than, for example, 15 cm. Moreover, the axial length of any one extension 1304 can be either similar to, or independent from, the axial length of any other extension 1304. Each extension 1304 can have an axial length of, for example, at least 1 mm, at least 2 mm, at least 5 mm, at least 1 cm, at least 2 cm, at least 3 cm, at least 4 cm, at least 5 cm, at least 6 cm, at least 7 cm, at least 8 cm, at least 9 cm, at least 10 cm, at least 11 cm, at least 12 cm, at least 13 cm, at least 14 cm, at least 15 cm, or another suitable length. In some embodiments, each extension 1304 can have an axial length of no more than 20 cm, no more than 15 cm, no more than 10 cm, no more than 5 cm, no more than 2 cm, no more than 1 cm, no more than 5 mm, or another suitable length. The catheter 1300 can be implanted so that the distal end portion 1302 and each of the extensions 1304 are situated in the space between the renal capsule and the renal cortex. The catheter 1300 can then be anchored in place. FIG. 13 illustrates an embodiment in which the catheter 1300 can be anchored (e.g., to the renal capsule) using one or more sutures 818. In alternative embodiments, the catheter 1300 can be anchored using a balloon (e.g., as described with respect to the embodiment shown in FIG. 10).

FIG. 14 illustrates an embodiment in which the therapy-delivery module 702 can include a plurality of reservoirs 1400, 1400′ and a plurality of pumps 1402, 1402′ and the therapy-delivery apparatus 714 can include a plurality of catheters 1404, 1404′ with each catheter 1404, 1404′ having a distal end portion 1405, 1405′ having a plurality of extensions 1406, 1406′ each having one or more openings 1408, 1408′. In such embodiments, it can be possible to control the delivery of two or more drugs to the renal capsule independently of one another. This can be appropriate where, for example, one drug is administered in response to a minor degree or type of condition (e.g., minor hypertension or heart failure) and another drug is administered in response to more major degree or type of condition (e.g., major hypertension or heart failure). Alternatively, infusion of different drugs can be indicated in circumstances in which different discrete causes of a condition (e.g., hypertension or heart failure) are known and different drugs are indicated for treating the various discrete causes of the condition. If the same drug is provided to the two or more reservoirs 1400, 1400′, such an embodiment also can permit delivery of one drug, for example, to two different regions, at two different doses, at two different infusion rates, and/or at two different times.

In the embodiment shown in FIG. 14, one pump 1402, 1402′ can control delivery of a drug from one reservoir 1400, 1400′ through one catheter 1404, 1404′. In alternative embodiments, however, the device can be designed to incorporate, independently, various combinations of embodiments described above. For example, one pump 1402, 1402′ can control delivery of a drug from a plurality of reservoirs 1400, 1400′. Similarly, one reservoir 1400, 1400′ can dispense drug through a plurality of catheters 1404, 1404′. It is also possible that a plurality of reservoirs 1400, 1400′ can dispense drug through a single catheter 1404, 1404′. In embodiments in which a plurality of drugs can be infused through a single catheter 1404, 1404′, the outlet can include a one-way valve to reduce the likelihood and/or extent of backflow from the catheter 1404, 1404′ into the reservoir 1400, 1400′ and to limit the extent to which each reservoir 1400, 1400′ may be contaminated with drug stored in another reservoir 1400, 1400′. It is also possible that each catheter 1404, 1404′ can include a different design for increasing the number of openings 1408, 1408′. For example, one catheter 1404, 1404′ can include a patch while another catheter 1404, 1404′ can include a mesh sheet or a plurality of extensions 1406, 1406′. Furthermore, instead of having separate discrete catheters 1404, 1404′ as shown in FIG. 14, a device can include a catheter 1404, 1404′ that possess multiple lumens, where each lumen includes one or more openings 1408, 1408′ and/or distal structures (e.g., patches, mesh sheets, extensions, etc.).

### Selected Examples of Treatments

Some embodiments of the present technology can be used in the treatment of hypertension, heart failure, or another condition affected by renal activity. Generally, the method can include providing to a patient a drug-delivery system. As described above, certain components of the system can be implantable (e.g., an implantable medical device). Other components can be implantable or not implantable (e.g., a blood-pressure sensor). An implantable medical device can be implanted into the chest, abdominal cavity, or another suitable anatomical location of the patient. The implantable medical device can be implanted so that one or more catheters are positioned to deliver one or more drugs (e.g., an RAAS-suppressing drug) to a kidney of the patient. In some embodiments, a catheter can be positioned so that an opening through which the drug or drugs exit the catheter is positioned deep within the renal capsule (e.g., in the space between the renal capsule and the renal cortex).

Drug-delivery systems configured in accordance with embodiments of the present technology can be configured to determine if hypertension, heart failure, or another condition affected by renal activity is present in a patient. Responsive to the determination, or to another diagnosis of hypertension, heart failure, or another condition affected by renal activity, the system can be configured to automatically deliver a drug on a continuous or periodic basis. The system can use computer instructions (e.g., executed by a processor) to determine when to deliver a dosage of a drug to the renal capsule, renal arteries, or other tissue surrounding the kidney.

Drug-distribution patterns in accordance with embodiments of the preset technology can be influenced, in part, by flow rate, as described in more detail below. In some embodiments, it can be desirable to deliver drug to one or more selected areas of the kidney. Moreover, it can be desirable to place the catheter (or catheters or distal structures of one or more catheters) in one or more selected locations in the kidney. As discussed above, a physiological sensor (e.g., a blood-pressure sensor) can be implantable or can be extracorporeal. In either case, the physiological sensor can be configured to communicate (e.g., directly or indirectly) information regarding a physiological value (e.g., a blood-pressure value) of the patient to the implantable medical device. If the information transmitted to the implantable medical device indicates that drug should be administered to the patient, then the implantable medical device can respond to the information and administer the drug to the patient.

The parameters of administering the drug, such as drug identity (e.g., if the device contains more than one drug), dosing, frequency, timing, and/or location (e.g., if the device includes more than one catheter leading to more than one location) can be preprogrammed into a control module of the device. FIG. 15 is a block diagram illustrating an example of an algorithm including a set of instructions for the system. As described above with reference to FIG. 7, the algorithm can be stored on memory and be executed by a processor or another suitable component of the system. A clinician using step-by-step instructions may also implement the algorithm manually. With respect to the algorithm shown in FIG. 15, information regarding the patient's blood pressure can be monitored and the infusion rate of the drug adjusted according to predetermined parameters. Other analytical and/or control schemes are possible (e.g., analytical and/or control schemes based on physiological parameters other than blood pressure). The analytical and/or control schemes can be individualized for a patient and/or can be influenced by the number, identity, and/or activity of the drug or drugs provided in the device.

In some embodiments, the control module can be programmed to administer a predetermined dose of a drug to the patient when the physiological sensor senses a predetermined physiological value indicative of heart failure. In these and other embodiments, the control module can be programmed to administer varying doses of a drug depending upon the physiological value sensed by the physiological sensor. In such embodiments, the control module can be programmed, for example, to administer a first predetermined dose of a drug to the patient when the physiological sensor senses a first predetermined physiological value and to administer a second predetermined dose of the drug to the patient when the physiological sensor senses a second predetermined physiological value. Thus, the system can provide a graded response to a condition (e.g., hypertension or heart failure) that depends, at least to some extent, on the degree of the condition detected by the physiological sensor.

The system can be configured to provide a first response to a first condition or degree of severity and a second response to a second condition or degree of severity. For example, the first condition can be a chronic condition and the first response can be a maintenance response. Similarly, the second condition can be an acute condition (e.g., an emergency condition) and the second response can be a rescue response. A maintenance response can include delivering a maintenance drug or dosage, such as a drug or dosage indicated or otherwise well suited for frequent (e.g., daily) use. A rescue response can include delivering a rescue drug or dosage, such as a drug or dosage indicated or otherwise well suited for infrequent (e.g., one-time) use. In some embodiments, a maintenance drug or dosage is selected to have a slower and/or lesser effect on a condition (e.g., a physiological parameter associated with a condition) than a rescue drug or dosage.

In some embodiments, the system can provide a graded response to heart failure or another condition by varying the drug that is delivered in response to a physiological sensor detecting a hypertensive blood-pressure value. For example, the implantable medical device can include a first reservoir containing a first drug and a second reservoir containing a second drug. In these embodiments, the control module can be programmed to administer a predetermined dose of the first drug when the physiological sensor senses a first predetermined blood-pressure value and, alternatively, to administer a predetermined dose of the second drug when the physiological sensor senses a second predetermined blood-pressure value.

Drug delivery in response to blood pressure or another suitable physiological parameter can occur in real time, near real time, or after a suitable delay. In some embodiments, a delay between measurement of a physiological parameter and drug delivery can correspond to an expected or observed delay between drug delivery and a physiological effect. For example, locally delivering an RAAS-suppressing drug to a kidney may not cause a corresponding effect on blood pressure for several hours or longer. The system can be configured to measure the physiological parameter after such a delay to determine whether the local drug delivery was effective, whether more of the drug should be delivered, and/or whether aspects of a drug-delivery schedule should be modified. With respect to the rate of infusion, low rates of infusion can tend to yield more equitable drug distribution radially. Faster flow rates can lead to broader drug distribution over more of the kidney than slower flow rates. As such, if it is desirable to reach a broad area of the kidney with the drug, the flow rate with which the drug is delivered can be increased. In such circumstances, it can be desirable to decrease the concentration of a drug to be delivered. If it is desired to have a drug localized to a particular region of the kidney, the flow rate with which the drug is delivered can be decreased. In such circumstances, it can be desirable to increase the concentration of the drug within a solution delivered through the catheter. Furthermore, in addition to or instead of delivering drugs, systems and methods configured in accordance with embodiments of the present technology can be used to introduce physical environmental changes to the kidney to modulate renal function. For example, one or more solutions can be infused that can alter, for example, the temperature of the kidney and, consequently, alter renal activity.

### EXPERIMENTAL EXAMPLE

The present disclosure is further illustrated by the following experimental example. It is to be understood that the particular materials, amounts, and procedures are to be interpreted broadly in accordance with the scope and spirit of the invention as set forth herein.

Spontaneously hypertensive rats (Charles River Laboratories International, Inc., Wilmington, Mass.) were housed individually in metabolic cages so that their urine could be captured. Pumps were implanted into the rats with catheters placed in the renal capsule to delivery drugs on a chronic basis with the categories of drugs according to Table 1.

TABLE 1
Drug Categories

| Group | Name |
|---|---|
| 1 | Surgery development |
| 2 | Renin inhibitor |
| 3 | Diuretic |
| 4 | Local anesthetic |
| 5 | High-concentration sodium solution |
| 6 | Calcium-channel blocker |
| 7 | Alpha2 receptor agonist |
| 8 | Angiotensin II receptor antagonist (vasal dilator) |
| 9 | Control |

Table 2 shows the procedural schedule. The drug dosages were selected so that the drugs would be delivered through Week 4. Thus, each animal had a period of drug administration followed by a period without the drug.

TABLE 2
Procedural Schedule

| | Time Point |
|---|---|
| Procedure | (relative to implant) |
| Implant | — |
| Urine collection | Prior to implant |
| | Week 2 |
| | Week 4 |
| | Week 5 |
| | Term |
| Blood collection | Prior to implant |
| | Week 2 |
| | Week 4 |
| | Week 5 |
| | Term |
| Blood pressure | Prior to implant, |
| measurements | 2x/week each week to term |
| Termination | Up to 7 weeks |

In a laminar flow hood, using sterile technique, reservoirs of the pumps were filled with the drugs according to the instructions provided by the pump manufacturer. The filled pumps were surgically placed into the rats using standard surgical procedures. A 1.5 cm to 2.0 cm incision was made along the left dorsal aspect of the abdomen just caudal to the last rib to access the left kidney. The left kidney was isolated and 7-0 PROLENE, monofilament, nonabsorbable polypropylene suture (Ethicon, Inc., Somerville, N.J.) was used to make a purse string in the capsule. A catheter was placed via a flank approach for both kidneys in each animal, entering behind the last rib on the dorsal aspect of the abdomen. Following purse-string placement, the catheter was placed under the capsule through a small incision made at the center of the purse string. The 7-0 PROLENE purse string was then tightened, and a Chinese finger trap suture technique was used to secure the catheter. Catheter placement was repeated for the right kidney, with the sides reversed and using a slightly more caudal incision. A pocket was made cranial to the laparotomy incision just to the left of midline. A filled pump was placed in the pocket and connected to the catheter placed to the left kidney. All incisions were closed in a standard fashion. Pump placement was repeated for the right kidney with the sides reversed.

Urine was collected from the metabolic cages as scheduled in Table 2. Urine samples were centrifuged 24 hours after collection to remove particulate matter. The samples were centrifuged for 3-5 minutes at a low rpm. Three 1 mL samples were aliquoted and frozen at −80° C. for later analysis. Samples were analyzed for drug content using commercially available methods. Blood for biological markers was drawn from the jugular vein or alternative vessel prior to the day of surgery. The animals were lightly anesthetized with isoflurane. Plasma was collected using EDTA (ethylenediaminetetraacetic acid) as an anticoagulant. Samples were centrifuged for 15 minutes at 1000×g at 2° C. to 8° C. within 30 minutes of collection and the plasma was removed. The plasma was assayed immediately or stored at −80° C. for later analysis. Biological markers (e.g., renin, norepinephrine, angiotensin, etc.) were detected and/or quantified using commercially available methods. Blood pressure was measured via a tail-cuff (CODA Blood Pressure Monitor system, Kent Scientific Corp., Torrington, Conn.) to assess the affect of the drug on blood pressure. At termination, the animals were heparinized and euthanized using standard procedures and the kidneys harvested. For each animal, the catheterized kidney was exposed, removed intact, and an image captured of the capsular surfaces. Then, the catheter was removed and/or cut, and both kidneys were individually weighed with the capsule intact, but after removal of any excess fat, vessels, and connective tissue.

Drugs were delivered by loading into a 2ML1 ALZET minipump (Durect Corp., Cupertino, Calif.) for which the rate of elution was 240 μL/day. The predetermined amount of drug was dissolved in solvent as shown in Table 3 and filtered through a 0.22 μm filter. Controls include only solvent.

TABLE 3
Drugs

| | | Maximum | | | |
|---|---|---|---|---|---|
| | | Dosage | Concentration | Intended | |
| Drug | Drug Source | (mg/kg/day) | (mg/ml) | Effect | Solvent |
| aliskiren¹ | Novartis | 0.18 | 0.763 | renin inhibitor | 0.9% saline |
| | | | | | dilution |
| chlorothiazide² | Sigma C4911 | 0.37 | 1.526 | diuretic | 0.9% saline |
| indapamide | Sigma I1887 | 0.04 | 0.153 | diuretic | 15% ethanol |
| | (powder) | | | | |
| lidocaine | Sigma L7757 | 0.04 | 0.153 | block local | 0.9% saline |
| | | | | neural signals | (dilution) |
| procaine | Sigma P9879 | 0.04 | 0.153 | block local | 0.9% saline |
| | | | | neural signals | (dilution) |
| high | 3x-10x NaCl | concentration | concentration | hypertonic | deionized |
| concentration | concentration | dependent | dependent | solution | sterilized |
| NaCl solution | (0.9% saline | | | | H2O |
| | baseline) | | | | |
| amlodipine³ | Sigma A5605 | 0.04 | 0.153 | calcium- | 0.9% saline |
| | (solid) | | | channel blocker | |
| losartan⁴ | Sigma 61188 | 0.07 | 0.305 | angiotensin II | 0.9% saline |
| | | | | receptor | |
| | | | | antagonist | |
| clonidine⁵ | Sigma C7897 | 0.01 | 0.023 | alpha² receptor | 0.9% saline |
| | | | | agonists | |

¹TEKTURNA (Novartis Pharmaceuticals Corporation, East Hanover, NJ)
²DIURIL (Salix Pharmaceuticals, Inc., Morrisville, NC)
³NORVASC (Pfizer Inc. New York, NY)
⁴HYZAAR (Merck & Co., Inc., Whitehouse Station, NJ)
⁵CATAPRES (Boehringer Ingelheim Pharmaceuticals, Inc., Ridgefield, CT)

For each tested drug, an anti-hypertension effect is expected to be achieved at a lower dose than an oral dose necessary to achieve the same anti-hypertension effect using the same drug. Furthermore, local drug delivery to the kidneys is expected to result in lower serum renin and lower serum angiotensin concentrations in the treated animals relative to the untreated control animals. The results showed that, over time, the, systolic blood pressure, diastolic blood pressure, and mean blood pressure were reduced. Furthermore, heart rate was not reduced, although reduction in heart rate is widely reported as a side effect of clonidine. This result suggests that local drug delivery to the kidneys can reduce certain side effects associated with systemic drug delivery. Furthermore, the dosage of clonidine resulting in the blood-pressure effect was significantly less than an oral dosage that would be expected for a similar result.

### EXAMPLES

1. A system comprising:
a physiological sensor;
an implantable medical device including a reservoir configured to contain a drug and a catheter that includes a lumen extending between the reservoir and a delivery opening, wherein the implantable medical device is configured to be surgically implanted in a human patient with the delivery opening at or near a renal capsule of a kidney of the patient; and
a control module configured to communicate with the physiological sensor and to control delivery of the drug through the delivery opening in response to a physiological parameter measured by the physiological sensor.

2. The system of example 1 wherein the delivery opening is at a distal end of the catheter.

3. The system of example 1 wherein:
the implantable medical device further includes a pump operably connected to the reservoir; and
the control module is configured to control delivery of the drug by controlling operation of the pump.

4. The system of example 1 wherein the physiological parameter is blood pressure.

5. The system of example 1 wherein the reservoir includes a self-sealing inlet.

6. The system of example 1 wherein the physiological sensor includes an implantable sensor.

7. The system of example 1 wherein the physiological sensor includes an extracorporeal device.

8. The system of example 7 wherein the control module is configured to communicate with the extracorporeal device wirelessly.

9. The system of example 1 wherein the implantable medical device further includes a distal structure having a plurality of delivery openings.

10. The system of example 9 wherein the distal structure includes a patch, a mesh sheet, an extension, or a combination thereof.

11. The system of example 1 wherein:
the reservoir is a first reservoir;
the drug is a first drug; and
the implantable medical device further includes a second reservoir configured to contain a second drug.

12. The system of example 11 wherein:
the implantable medical device further includes a first pump operably connected to the first reservoir and a second pump operably connected to the second reservoir;
the control module is configured to control delivery of the first drug by controlling operation of the first pump; and
the control module is configured to control delivery of the second drug by controlling operation of the second pump.

13. The system of example 11 wherein:
the first and second reservoirs are fluidly connected to the lumen; and
the implantable medical device further includes—
   a first check valve that prevents fluid flow from the lumen to the first reservoir; and
   a second check valve that prevents fluid flow from the lumen to the second reservoir.

14. The system of example 11 wherein:
the catheter is a first catheter;
the delivery opening is a first delivery opening;
the lumen is a first lumen; and
the implantable medical device further includes a second catheter including a second lumen extending between the second reservoir and a second delivery opening.

15. The system of example 1 wherein the implantable medical device further includes a connector configured for connection to the renal capsule.

16. The system of example 15 wherein the connector includes a suture site.

17. The system of example 15 wherein the connector includes a balloon.

18. The system of example 17 wherein:
the balloon is a first balloon;
the connector further includes a second balloon; and
the connector is configured to squeeze the renal capsule between the first and second balloons.

19. A method of treating a human patient, comprising:
delivering a drug to a delivery site at or near a renal capsule of a kidney of the patient via an implanted medical device;
measuring a physiological parameter in the patient corresponding to a condition affected by renal activity; and
automatically controlling delivery of the drug in response to the physiological parameter to treat the condition.

20. The method of example 19 wherein the drug is a diuretic, an aldosterone antagonist, a vasodilator, a renin inhibitor, or a combination thereof.

21. The method of example 19 wherein the drug is bumetanide, furosemide, a natriuretic peptide, or a combination thereof.

22. The method of example 19 wherein the drug is spironolactone, eplerenone, or a combination thereof.

23. The method of example 19 wherein the drug is isosorbide, isosorbide dinitrate, isosorbide-5-mononitrate, apresoline, or a combination thereof.

24. The method of example 19 wherein the drug is aliskiren.

25. The method of example 19 wherein the drug is clonidine.

26. The method of example 19 wherein the delivery site is generally within a potential space of the kidney outside the vasculature of the kidney.

27. The method of example 19 wherein the delivery site is between thy, renal capsule and a cortex of the kidney.

28. The method of example 19 wherein the physiological parameter is blood pressure.

29. The method of example 19 wherein:
the patient has diagnosed hypertension; and
the method further comprises improving the physiological parameter and/or another physiological parameter corresponding to the diagnosed hypertension.

30. The method of example 19 wherein:
the patient has diagnosed heart failure; and
the method further comprises improving the physiological parameter and/or another physiological parameter corresponding to the diagnosed heart failure.

31. The method of example 19 wherein automatically controlling delivery of the drug includes automatically controlling operation of a pump of the implanted medical device.

32. The method of example 19 wherein:
measuring the physiological parameter includes measuring the physiological parameter using an extracorporeal device; and
the method further comprises wirelessly communicating the physiological parameter to the implanted medical device.

33. The method of example 19 wherein measuring the physiological parameter includes measuring the physiological parameter using an implantable sensor.

34. The method of example 19 wherein automatically controlling delivery of the drug includes delivering different selected dosages of the drug in response to the physiological parameter.

35. The method of example 19 wherein:
the drug is a first drug;
the delivery site is a first delivery site; and
the method further comprises delivering a second drug to a second delivery site at or near the renal capsule via the implanted medical device.

36. The method of example 19 wherein:
measuring the physiological parameter includes measuring the physiological parameter continuously and/or intermittently over a period of time to generate physiological data;
the method further comprises generating a representation of the physiological data; and
automatically controlling delivery of the drug includes automatically controlling delivery of the drug in response to the representation.

37. The method of example 36 wherein the representation is an average.

38. The method of example 19 wherein:
the drug is a first drug;
the method further comprises delivering a second drug to the delivery site; and
automatically controlling delivery of the first drug includes delivering the first drug or the second drug in response to the physiological parameter.

39. The method of example 38 wherein:
the first drug is a maintenance drug; and
the second drug is a rescue drug.

40. The method of example 39 wherein:
the maintenance drug is an aldosterone antagonist, a vasodilator, a renin inhibitor, or a combination thereof; and
the rescue drug is a diuretic.

41. The method of example 19, further comprising anchoring a portion of the implanted medical device to the renal capsule.

42. The method of example 41 wherein anchoring includes stitching a suture site of the implanted medical device to the renal capsule.

43. The method of example 41 wherein anchoring includes inflating or positioning a balloon proximate the renal capsule.

44. The method of example 41 wherein anchoring includes inflating or positioning a first balloon proximate a first side of the renal capsule and inflating or positioning a second balloon proximate a second side of the renal capsule.

### Conclusion

The above detailed descriptions of embodiments of the present technology are for purposes of illustration only and are not intended to be exhaustive or to limit the present technology to the precise form(s) disclosed above. Various equivalent modifications are possible within the scope of the present technology, as those skilled in the relevant art will recognize. For example, while stages may be presented in a given order, alternative embodiments may perform stages in a different order. The various embodiments described herein and elements thereof may also be combined to provide further embodiments. In some cases, well-known structures and functions have not been shown or described in detail to avoid unnecessarily obscuring the description of embodiments of the present technology.

Where the context permits, singular or plural terms may also include the plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

I/We claim:

1. A system comprising:
a physiological sensor;
an implantable medical device including a reservoir configured to contain a drug and a catheter that includes a lumen extending between the reservoir and a delivery opening, wherein the implantable medical device is configured to be surgically implanted in a human patient with the delivery opening at or near a renal capsule of a kidney of the patient; and
a control module configured to communicate with the physiological sensor and to control delivery of the drug through the delivery opening in response to a physiological parameter measured by the physiological sensor.

2. The system of claim 1 wherein the delivery opening is at a distal end of the catheter.

3. The system of claim 1 wherein:
the implantable medical device further includes a pump operably connected to the reservoir; and
the control module is configured to control delivery of the drug by controlling operation of the pump.

4. The system of claim 1 wherein the physiological parameter is blood pressure.

5. The system of claim 1 wherein the reservoir includes a self-sealing inlet.

6. The system of claim 1 wherein the physiological sensor includes an implantable sensor.

7. The system of claim 1 wherein the physiological sensor includes an extracorporeal device.

8. The system of claim 7 wherein the control module is configured to communicate with the extracorporeal device wirelessly.

9. The system of claim 1 wherein the implantable medical device further includes a distal structure having a plurality of delivery openings.

10. The system of claim 9 wherein the distal structure includes a patch, a mesh sheet, an extension, or a combination thereof.

11. The system of claim 1 wherein:
the reservoir is a first reservoir;
the drug is a first drug; and
the implantable medical device further includes a second reservoir configured to contain a second drug.

12. The system of claim 11 wherein:
the implantable medical device further includes a first pump operably connected to the first reservoir and a second pump operably connected to the second reservoir;
the control module is configured to control delivery of the first drug by controlling operation of the first pump; and
the control module is configured to control delivery of the second drug by controlling operation of the second pump.

13. The system of claim 11 wherein:
the first and second reservoirs are fluidly connected to the lumen; and
the implantable medical device further includes—
a first check valve that prevents fluid flow from the lumen to the first reservoir; and
a second check valve that prevents fluid flow from the lumen to the second reservoir.

14. The system of claim 11 wherein:
the catheter is a first catheter;
the delivery opening is a first delivery opening;
the lumen is a first lumen; and
the implantable medical device further includes a second catheter including a second lumen extending between the second reservoir and a second delivery opening.

15. The system of claim 1 wherein the implantable medical device further includes a connector configured for connection to the renal capsule.

16. The system of claim 15 wherein the connector includes a suture site.

17. The system of claim 15 wherein the connector includes a balloon.

18. The system of claim 17 wherein:
the balloon is a first balloon;
the connector further includes a second balloon; and
the connector is configured to squeeze the renal capsule between the first and second balloons.

19. A method of treating a human patient, comprising:
delivering a drug to a delivery site at or near a renal capsule of a kidney of the patient via an implanted medical device;
measuring a physiological parameter in the patient corresponding to a condition affected by renal activity; and
automatically controlling delivery of the drug in response to the physiological parameter to treat the condition.

20. The method of claim 19 wherein the drug is a diuretic an aldosterone antagonist, a vasodilator, a renin inhibitor, or a combination thereof.

21. The method of claim 19 wherein the drug is bumetanide, furosemide, a natriuretic peptide, or a combination thereof.

22. The method of claim 19 wherein the drug is spironolactone, eplerenone, or a combination thereof.

23. The method of claim 19 wherein the drug is isosorbide, isosorbide dinitrate, isosorbide-5-mononitrate, apresoline, or a combination thereof.

24. The method of claim 19 wherein the drag is aliskiren.

25. The method of claim 19 wherein the drag is clonidine.

26. The method of claim 19 wherein the delivery site is generally within a potential space of the kidney outside the vasculature of the kidney.

27. The method of claim 19 wherein the delivery site is between the renal capsule and a cortex of the kidney.

28. The method of claim 19 wherein the physiological parameter is blood pressure.

29. The method of claim 19 wherein:
the patient has diagnosed hypertension; and
the method further comprises improving the physiological parameter and/or another physiological parameter corresponding to the diagnosed hypertension.

30. The method of claim 19 wherein:
the patient has diagnosed heart failure; and
the method further comprises improving the physiological parameter and/or another physiological parameter corresponding to the diagnosed heart failure.

31. The method of claim 19 wherein automatically controlling delivery of the drug includes automatically controlling operation of a pump of the implanted medical device.

32. The method of claim 19 wherein:
measuring the physiological parameter includes measuring the physiological parameter using an extracorporeal device; and
the method further comprises wirelessly communicating the physiological parameter to the implanted medical device.

33. The method of claim 19 wherein measuring the physiological parameter includes measuring the physiological parameter using an implantable sensor.

34. The method of claim 19 wherein automatically controlling delivery of the drug includes delivering different selected dosages of the drug in response to the physiological parameter.

35. The method of claim 19 wherein:
the drug is a first drug;
the delivery site is a first delivery site; and
the method further comprises delivering a second drug to a second delivery site at or near the renal capsule via the implanted medical device.

36. The method of claim 19 wherein:
measuring the physiological parameter includes measuring the physiological parameter continuously and/or intermittently over a period of time to generate physiological data;
the method further comprises generating a representation of the physiological data; and
automatically controlling delivery of the drug includes automatically controlling delivery of the drug in response to the representation.

37. The method of claim 36 wherein the representation is an average.

38. The method of claim 19 wherein:
the drug is a first drug;
the method further comprises delivering a second drug to the delivery site; and
automatically controlling delivery of the first drug includes delivering the first drug or the second drug in response to the physiological parameter.

39. The method of claim 38 wherein:
the first drug is a maintenance drug; and
the second drug is a rescue drug.

40. The method of claim 39 wherein:
the maintenance drug is an aldosterone antagonist, a vasodilator, a renin inhibitor, or a combination thereof; and
the rescue drug is a diuretic.

41. The method of claim 19, further comprising anchoring a portion of the implanted medical device to the renal capsule.

42. The method of claim 41 wherein anchoring includes stitching a suture site of the implanted medical device to the renal capsule.

43. The method of claim 41 wherein anchoring includes inflating or positioning a balloon proximate the renal capsule.

44. The method of claim 41 wherein anchoring includes inflating or positioning a first balloon proximate a first side of the renal capsule and inflating or positioning a second balloon proximate a second side of the renal capsule.
